(19) United States
(12) Patent Application Publication
(10) Pub. No.: US 2009/0053989 A1
(43) Pub. Date: Feb. 26, 2009

(54) Interior Air Quality Space and Methods of Designing and Constructing Same

(76) Inventors: Tom Lunde, Beach Park, IL (US); Nick Nardella, Glen Ellyn, IL (US)

Correspondence Address: LAW OFFICES OF MARK A. HAMILL, P.C., 45 SOUTH PARK BLVD., SUITE 205, GLEN ELLYN, IL, 60137, US

(21) Appl. No.: 11/868,468

(22) Filed: Oct. 5, 2007

Related U.S. Application Data
(63) Continuation-in-part of application No. PCT/US2005/013381, filed on Apr. 6, 2005.

(51) Int. Cl. A61L 9/00; B23P 19/04; G01N 7/00; G01N 33/00

(52) U.S. Cl. 454/187; 29/700; 73/31.02; 422/83; 96/223

(57) ABSTRACT

The invention provides a method and system for constructing a new space or improving and existing space to achieve and maintain high air quality in the interior of the space by limiting airborne allergens, VOCs, particulates, and bio-aerosols therein. One method of the invention, includes the steps of sampling the air quality of the indoor space, removing suspected sources of pollutants, selecting replacement materials having low-VOC off gassing, testing the air quality to ensure the space meets a pre-determined base line air quality, and maintaining the air quality of the indoor space at or below the pre-determined base line air quality.

### FIELD OF THE INVENTION

The present invention is directed to interior spaces having improved air quality and methods of designing, constructing, and maintaining such spaces.

### BACKGROUND

At the present time, a large variety of chemicals are introduced into indoor environments at a rate of about 6,000 new formulas per year. When furnishings, building materials, and other interior products are produced, certain chemicals found in, for example, glues or epoxies, drying agents in paints, and molded plastics emit vapors into the surrounding air. This process is generally known as “off-gassing.” The process of “off gassing” typically pumps a significant amount of volatile organic compounds (VOCs) into the atmosphere. Another source of VOCs and other problematic chemicals are cleaning products, including chemicals use in the dry cleaning industry. The issue of indoor chemical air pollution has become an increasingly acute since many modems buildings are tightly sealed to reduce energy loss from escaping warmed or cooled air. As a result, the indoor air quality of many such sealed spaces, such as homes, office buildings, hotels and schools, is often lower than is desirable for optimal human health.

In addition to chemical gases discussed above, allergens are another major concern in indoor air quality. Allergens are particularly problematic for children and adults with allergies, asthma, or chemical sensitivity. Dust mites, family pets, cockroaches, rodents, and mold are just a few sources of allergens that can cause allergic reactions in susceptible populations. These potential particulate allergens can be found on the surfaces of an interior space and often become airborne. When a potential allergen or toxin is derived from a biological source, such as mold, dust mites or bacteria, and is small and fine enough to become easily airborne, it is defined as a “bio-aerosols.” Typical, interior bio-aerosols include pollen, insect parts, skin cells, fibers and microbial toxins and cell wall components. When bio-aerosols are inhaled into the human body, allergic reactions, respiratory problems, and illnesses may result. For these reasons, bio-aerosols are also a major factor to consider in improving indoor air quality.

Indoor mold growth is a problem that only now is being widely recognized and addressed. Thousands of distinct mold allergens are believed to exist and only a few have been characterized. Recent media attention has brought to light the dangers associated with exposure to mold, especially in the home environment. Causes of mold in the home include poor maintenance or high moisture in wood or paper veneer found on drywall. Both can combine to contribute to mold growth and can go unnoticed for years. A variety of mold species can also contribute a particularly troublesome class of bio-aerosols to the indoor air, namely, mycotoxins. These compounds are toxic chemicals or fine particulates released by mold and/or mold spores. Certain mycotoxins are currently believed to be capable of causing allergic reactions, respiratory problems and even illness in humans.

Across the United States, the number of people diagnosed with allergies and/or asthma has continued to increase over recent years. Most experts agree that improved indoor air quality can improve the health of the general population, and is particularly advantageous for those with chemical sensitivities, asthma, or allergies.

Another common problem in the hospitality industry is that designated non-smoking rooms or suites are frequently contaminated by illicit smoking of tobacco products by guests. This can lead to unwanted odors in the room or suites which can be a significant problem as subsequent non-smoking guests assigned to such rooms find the residual smoke odors offensive. Such situations are a significant problem for a hotel since extra cleaning is often necessary to remove the odors and the room is typical out of service until those steps can be taken. In some hotels, the problem has become so troublesome that fines are assessed against any guest caught illicitly smoking in a designated non-smoking area. Nonetheless, illicit smoking remains significant problem as it frequently difficult to determine whether it has occurred until there is a subsequent guest complaint. It would be desirable for the hospitality industry to have a means for determining when illicit smoking is occurring in a guest room, for preventing additional smoking in real-time, and for preventing future illicit smoking.

### SUMMARY

One embodiment of the present invention includes a method of improving the air quality of an existing interior space. The process includes the steps of sampling the interior space for chemical pollutants and other common allergen content; analyzing the chemical pollutant and other common allergen content of the samples; removing suspected sources of the allergens, chemical pollutants and bio-aerosols in the interior space; selecting replacement interior materials with chemical pollutant and other common allergen content below predetermined acceptable values; utilizing the selected materials within the interior space; testing samples of the completed interior space for chemical pollutant and other common allergen content to set an air quality base line for the interior space; maintaining the interior space in a manner which the chemical pollutant and other common allergen content are kept at or near the air quality baseline. Preferably, this method of invention includes the further step of monitoring the indoor air quality of the space on a continuous or a nearly continuous basis to provide real-time feedback for the facilities management staff as well as for training cleaning crews and maintenance personnel. Alternately, the process may also include the step of periodically testing the interior space samples for chemical pollutant and other common allergen content to evaluate whether the maintenance process has been effective in keeping the chemical pollutant, and other common allergen content of the space at or near the air quality baseline level. The method also preferably includes the step of selecting materials, for example, hardwood, tile or low nap carpeting for flooring material, that are not conducive to the growth of biological organisms (e.g., molds, dust mites, bacteria) that commonly produce potential allergens or bio-aerosols.

One alternative method of the invention further includes the step of sampling and analyzing for bio-aerosols. These steps are typically undertaken only after analysis of the common particulate allergen samples yield mold spore counts that are either abnormally high or that reveal the presence of certain problematic mold species which commonly produce mycotoxins. Under those circumstances additional testing should be preformed for the presence of bio-aerosols, such as mycotoxins. If they are present, then materials suspected of harboring the organism responsible for the production of the bio-aerosol should be removed and/or remediated. After reconstruction of the interior space, the space is preferably tested again for bio-aerosol content and a bio-aerosol baseline is established.

In the methods of the invention, an important step in maintaining the low chemical pollutant, bio-aerosol, and other common allergen content baseline of an interior space is to install, utilize and properly maintain a suitable air purification system for the interior space. This typically will include the use of air purification system that includes redundant air purification sub-systems. For example, a particularly preferred air purification system includes an ion pulse generator, ozone generator (for use in sanitation mode), a dust filter, and may also include a high efficiency particulate air filter (“HEPA filter”) in the air handling system for the space. The filters, pulse generator and/or ozone generator may be part of the HVAC system for the interior space or it may be in the form of a separate air filtration unit. In the many instances where the outdoor environment near the indoor space is relatively chemically pollutant free, the indoor chemical air content testing can be largely directed at measuring and monitoring total VOC content of the indoor space.

In the method set forth above, the step of maintaining the interior space at or near the baseline preferably also includes the steps of training maintenance staff and/or cleaning staff with best practices for doing so including; changing filters on the air purification systems for the interior space; vacuuming the space with HEPA filtered vacuum cleaner; periodic cleaning of any duct work within the space; cleaning floors bathrooms, windows and other surface with appropriate cleaning supplies (low VOC and lacking chemical irritants); utilizing low VOC, low allergen content detergents for towels, linens and other items which are regularly laundered.

Further, the method preferably includes the steps of field testing at least some of the materials to be brought into the interior space to ensure that they meet the manufacturers' specifications for low VOC off gassing and being free of common chemical irritants. This step is important for materials that have a large surface area or are used in large volumes in the space to ensure that the VOC level in the interior space can be maintained at or near an acceptable baseline level. This analysis process for the materials is not limited to the material itself, but includes all of the chemical compounds used therein such as adhesives, laminates, varnishes, paint, tints, etc. Whenever possible materials are selected which utilize water soluble adhesives, paints, etc, to attempt to minimize the VOC content of the interior space. Preferably, this method of the invention also includes the step of consulting with furniture, carpeting and other interior material manufacturers to assist in the selection of the chemicals and materials used in manufacturing the furniture, carpeting and other interior furnishing. This process would include exclusion of chemicals and resins which are typically used in the manufacture of such articles as well as the selection of materials which are not conducive to the growth of organisms, such as dust mites, bacteria and molds, which constituted common allergens or which commonly generate bio-aerosols.

Another method of the invention includes a method of constructing a low VOC, low allergen, and low bi-aerosol interior space. The process includes the steps of selecting interior buildings materials which total VOC off gassing is less than about 0.5 mg/m3, selecting structural supports which total VOC off gassing is less than about 0.5 mg/m3; selecting interior wall materials which total VOC off gassing is less than about 0.5 mg/m3; selecting flooring materials which total VOC off gassing is less than 0.5 mg/m3; and selecting furnishings which total VOC off gassing is less than about 0.5 mg/m3. It is preferred that the off gassing limits set forth in this preferred method are measured in accordance with ASTM Standard D-5116-97 and D-6670-01. In this method of invention, it is preferred that each of the components materials used in constructing the interior space is analyzed for chemical inertness and both short and long term off gassing of VOCs. Preferably, the methods of the invention further includes the steps of testing the constructed interior space to set a base line for VOC content and training staff to maintain the air quality of the space at or near that baseline.

The invention further includes an interior space having improved air quality comprising: structural supports selected to total VOC off gassing is less than about 0.5 mg/m3; interior wall materials selected to total VOC off gassing is less than about 0.5 mg/m3; flooring materials selected to total VOC off gassing is less than about 0.5 mg/m3; and furnishings selected to total VOC off gassing is less than about 0.5 mg/m3. In the interior space of the invention, each of the components materials used in constructing the interior space are analyzed for chemical inertness and long term VOC off gassing. This analysis process is not limited to the material itself, but includes all of the chemical compounds used therein such as adhesives, laminates, varnishes, paint and tints which are used in its construction. Preferably, the flooring material includes structural sub-flooring such as low VOC plywood, cement, or cork (as a sub-flooring material), as well as low VOC decorative surface materials such as tiles, low nap carpeting, hardwood, etc.

In yet another alternate embodiment of the invention, a system of constructing and maintaining the indoor air quality of an interior space includes the steps of: selecting construction materials with chemical pollutant and other common allergen content below predetermined acceptable values; constructing the space utilizing the selected materials within the interior space; near continuous monitoring of the indoor air quality of the completed interior space for chemical pollutant and/or other common allergens; analyzing the indoor air quality data for elevated levels on chemical pollutants and/or other common allergens; and remediating any suspected sources of elevated levels of chemical pollutants and/or other common allergens. Preferably, this embodiment of the invention includes the step of utilizing best indoor air quality maintenance practices to minimize the levels of chemical pollutants and/or common allergens. The invention may further include the step of training maintenance staff to utilize best indoor air quality maintenance practices and to evaluate the results of such training based at least partially on the basis of the indoor air quality data. The method may still further include the steps of retraining the maintenance staff in view of the indoor air quality data and/or modifying the best indoor air quality maintenance practices in view of the indoor air quality data.

Another alternate method of the invention includes the steps of near continuous monitoring of the indoor air quality of a guest space for byproducts of tobacco smoking; analyzing the indoor air quality data for tobacco smoking byproducts above a predetermined level; generating a signal when the presence of one or more tobacco smoke byproducts exceeds the predetermined level; and communicating that signal to facilities operations staff to notify them that a guest is suspected to be illicitly smoking in a non-smoking guest room. In one preferred embodiment, the method includes a further step of generating a record of the suspected elicit smoking event, matching the record to a database containing information concerning the guest currently occupying the room in question, and querying the database for prior instances of suspected illicit smoking. The method may also include the further step of charging the account of the suspected illicitly smoking guest for an additional room cleaning charge to removing any unpleasant orders and/or banning the suspected illicitly smoking guest in question from occupying non-smoking rooms in the future.

A further embodiment of the invention includes a system for maintaining the indoor air quality of an interior space comprising, a real-time indoor air quality monitoring sensor capable of detecting air quality data which is indicative of the presence or absence of a human being within the interior space, and air purification system operably connected to the real-time indoor air quality sensor, the indoor air quality purification system having at least one sanitizing mode of operation during which the presence of humans in the interior space is undesirable, and a switching component for selectively activating and the de-activating the sanitizing mode of the air quality purification system in response to the air quality data which is indicative of the presence or absence of humans within an interior space. The preferred switching component of the system is preferably a microprocessor operably linked to the air quality monitoring sensor, but may also be a mechanical switch. It is also prefer that the air quality purification system include at least one gentler, air purification mode in which humans may be present within the interior space during operation of the mode. Preferably, the air purification system of this embodiment of the invention includes an ozone generator which may be switched between sanitizing and purification modes. The low ozone output mode which would be at a level that is more appropriate for human occupation of the interior space during operation, but which would be less efficient at removing airborne contaminants from the space than in the sanitizing mode. In this way, after an interior space is vacated by its human occupants, the air purification system can automatically be switched to sanitation mode so that odors, airborne particles, bacteria, mold, viruses can be neutralized by the system.

### BRIEF DESCRIPTION OF THE DRAWINGS

The organization and manner of the structure and function of the invention, together with the further objects and advantages thereof, may be understood by reference to the following description taken in connection with the accompanying drawings, and in which:

FIG. 1 is a schematic view of the heating system in accordance with one preferred embodiment of the invention;

FIG. 2 is an air intake and exhaust unit in accordance with the embodiment of the invention of FIG. 1;

FIG. 3 is a schematic view of ventilation system in accordance with the embodiment of FIG. 1;

FIG. 4 is an airwere filtration system in accordance with the Fonseca embodiment of invention of FIG. 1;

FIG. 5 is a schematic illustration of an air monitoring system in accordance with one embodiment of the invention; and,

FIG. 6 is a schematic illustration of a sensor array of the air monitoring system of FIG. 5.

### DETAILED DESCRIPTION OF THE ILLUSTRATED EMBODIMENT

In one method of the invention, an existing interior space of a building is converted to an allergy friendly space having low allergen, low VOC, and low bio-aerosol content. One of the initial steps in the process is to create an interior design plan in which appropriate materials have been selected for reconstruction of the space. This step in the process should include analysis of each of the materials to be used in the interior space for its contribution of common allergens, low VOC off gassing, low chemical irritant content and potential to facilitate the growth of biological organisms that generate common potential allergens and/or bio-aerosols. This process will typically be undertaken by having a Certified Industrial Hygienist (CIH) or similarly trained person. The review should preferably include each of the materials used in the interior space such as plastic resins, pressed wood products, structural wood products, metallic products, dry wall materials, joint compounds, textiles, quarried materials, wood finishes, adhesives and ceramics. The review should cover, not only all of the building materials used to reconstruct the space, but also the furnishings, bedding, window treatments, carpeting and accessories that will be placed therein.

One chemical irritant of particular interest is formaldehyde which is commonly used in pressed wood products, mold plastics products, plywood, sealants, foam mattresses, building insulation, and upholstery stuffing. Formaldehyde has been reported to cause eye, nose and throat irritations, and has been listed by governmental agencies as a possible carcinogen. Furthermore, formaldehyde can cause chemically sensitive persons to experience severe skin or respiratory symptoms. Another compound which causes similar health concerns is phenylcyclohexene. This compound is an off gassing product of many common glues and adhesives. Toluene is another chemical compound which should be avoided in the interior spaces of the invention. Toluene is a highly volatile liquid which is commonly found in oil based paints, inks, resins, and solvent based glues. In addition, dibutyl phthalates have also been identified as a suspected carcinogen and is used as an industrial solvent in a variety of consumer products. Another class of chemical compounds of concern is chlorinated ethylenes, such as percloroethylene, which are widely used in the dry cleaning industry. There are numerous other chemical compounds that can cause problems for chemically sensitive persons or those with asthma.

Other troubling compounds are commonly found in cleaning product for example, certain families of alkyl phenols, can mimic female estrogen hormones and are believed to be capable of interacting with the human endocrine system. These classes of chemicals are believed to interfere with human reproduction, and to increase the incidence of birth defects as well as breast, prostate and testicular cancers. A commonly used compound of this class is 4-nonylphenol which can be found in some detergents, disinfectants, and all-purpose cleaning agents. The United States National Toxicology Program (“NTP”) compiles lists of suspected carcinogens and known carcinogens. Generally, the compounds listed on the NTP should be avoided, where possible, and minimized where elimination is not possible.

Turning to the selection of appropriate building materials, interior walls and ceilings can be either conventional gypsum drywall board sealed with a dry mix joint compound. All products to be used in the space, including the drywall board, are preferably tested in dynamic environmental chambers following ASTM standards D-5116-97 and D-6670-01. However, other testing protocols may be used such as the U.S. Environmental Protection Agency's testing protocol for furniture and/or the State of Washington's protocol for interior furnishings and construction materials. Manufactured products should be measured for emission levels, which must meet the indoor air concentrations listed herein within 5 days of unpackaging. Air concentrations are based on the product being in a room 32 m³ in volume with an outdoor air concentration of 0.8 air changes per hour (ACH). Maximum allowable emission levels are preferably those set forth herein; however, they may also be those required by the state of Washington's indoor air quality program for new construction, the US Environmental Protection Agency's procurements specifications, the recommendations from the World Health Organization, or Germany's Blue Angel Program for electronic equipment. Listing of measured carcinogens and reproductive toxins are further identified by California Proposition 65 and the International Agency on Research on Cancer (IARC). Any pollutant not listed by those agencies should produce an air concentration level no greater than 1/10 the Threshold Limit Value (TLV) industrial work place standard (Reference: American Conference of Government Industrial Hygienists, 6500 Glenway, Building D-7, Cincinnati, Ohio 45211-4438). Further, any pollutant regulated as a primary or secondary outdoor air pollutant by the U.S. EPA should not be present in concentrations greater than that promulgated by the National Ambient Air Quality Standard (U.S. EPA, code of Federal Regulations, Title 40, Part 50). When multiple emission values are recommended in these alternate references, it is preferred that the lesser or more stringent should be used as the acceptable maximum emission value. The testing for measuring carcinogens and reproductive toxins preferably should identify levels for the most common indoor pollutant such as formaldehyde, total aldehydes, perchloroethylene, parardichlorobenzene, alkylphenols, ethoxylates, dibutyl phthalates and should at the very least include a measure of total VOCs. These compounds should also be avoided, where possible, or minimized where elimination is not possible.

Turning to specific construction materials and techniques, dry mix joint compound typically have fewer preservative chemicals than pre-mixed joint compound and are thus superior for the improved indoor space of the invention. The walls and ceilings are painted with low emission latex paint such as Gliden Lifemaster 2000™, or Eco Spec™ or Pristine™ from Benjamin Moore & Co. Of course other low emission paints may be used. Acceptable low emissions paints should meet, at a minimum, the following standards: total VOCs, 0.50 mg/m³; formaldehyde, 0.05 ppm; total aldehydes, 0.1 ppm; and styrene, 0.070 mg/m³. Alternately, blue board with a thin coat of veneer plaster may be applied without paint. Other joint compound or plaster alternatives include the use of the low emission primer and sealer products manufactured by American Formulating and Manufacturing (“AFM”) and sold under the trademark SAFECOAT.

The insulation in the exterior walls is preferably rock wool bats such as RSI 5.6 (R32) by Roxul, Inc. The use of rock wool batts is preferred because they have fibers of larger diameter than of other fibrous insulation materials such as fiberglass and are thus believed to be less likely to disperse particulate contaminants into the air. In moderate temperature zones, insulation with a lower R-value may be substituted. The exterior walls should also preferably include an air barrier such as Tyvek™ and a vapor barrier such as Poly Super 6™. Regardless of the type of insulation chosen, it should, at a minimum, meet the following standards: total VOCs, 0.50 mg/m³; formaldehyde, 0.05 ppm; total aldehydes, 0.1 ppm; and respirable particles, 0.05 mg/m³.

The preferred sub-flooring material is cement which is commonly used in steel frame construction. It is preferred that the cement surface be sealed with a low emission sealant to prevent water or gas vapors permeating through its relatively porous surface. Sealing the cement, whether on the floors of walls, is particularly advantageous when the interior space is in a basement or other areas where cement is in contact with the ground. Further, in such situations, it is important that the ground under the cement has good drainage to prevent migration of moisture through the cement to retard mold growth. The low emission sealants used should preferably meet the air quality measurement set forth for construction adhesives herein below.

In wood frame buildings, the preferred sub flooring is formaldehyde free plywood, such as AdvanTech™ Flooring. Such preferred plywood products are bonded with phenolic resins which have been shown in tests by U.S. Forest Products Laboratory and Oak Ridge National Laboratory to have negligible formaldehyde emissions. Other particle board materials can be used for the sub-flooring. However, traditional “particleboard,” which is used extensively in subflooring, cabinetry, shelving, countertop substrate, doors, and furniture, is inappropriate for this application. Suitable composite wood boards, that do not contain urea formaldehyde, include Sierra Pine's Medite II and Medex MDF. These are two the few types of medium-density fiberboard (MDF) that are formaldehyde-free. Other alternatives include agricultural-waste fiberboards, such as Wheatboard™ by Primeboard, Gridcore™ by PrimeBoard, Inc., Fiberboard 28™ by EnviroSafe 2000, and Pacific Northwest Fiber's “Tree Free Particleboard™.” Regardless of the low emission subflooring chosen, it should meet the following standards; total VOCs, 0.50 mg/m³; formaldehyde, 0.050 ppm; total aldehydes, 0.1 ppm; and 4-phenylcyclohexene, 0.0065 mg/m³, and styrene 0.070 mg/m³.

The preferred surface flooring materials are selected to be unfavorable for the growth of biological organisms that produce potential allergens or bio-aerosols. These considerations exclude the use of most long knap wall to wall carpeting. Examples of preferred surface flooring products include hardwood, tile or low nap carpeting for flooring material. Durable, inert ceramic flooring may also be preferred due to cost considerations, ease of cleaning, and low off gassing. The tile is preferably set on an acrylic modified set mortar, such as, MAPEI Ultra Flex Adhesive™ by MAPEI, Inc., and furnished with a low VOC grout such as MAPEI Kera Colour™ with Plastijoint™. Regardless of the materials chosen for use as a tile adhesive or any general construction adhesive in the interior space, it should meet the following standards: total VOCs, 0.50 mg/m³; formaldehyde, 0.050 ppm; total aldehydes, 0.1 ppm; and 4-phenylcyclohexene, 0.0065 mg/m³, and styrene 0.070 mg/m³. The preferred surface for placing the tile upon is cement bonded particle board such as Pyroc™. Hardwood is another option for the surface flooring material and can be finished and maintained with low-VOC emission materials. Another reason that hard wood or tile flooring is preferred is that they are generally compatible with radiant floor heating, which as set forth below may be preferred in many interior spaces of the invention. As improperly cleaned and maintained carpets can be good places for dust mites, bacteria and mold growth, if rugs are to be used, area rugs are preferable since they can be removed for more thorough cleaning. Further, carpeting and under padding made from natural fibers are also preferred since they generally do not outgas. Another reason to avoid wall to wall carpeting is that, when radiant floor heating is used, it tends to act as an insulator which can impair the efficiency of such heating systems. Regardless of the type of flooring chosen, it should meet the following standards: total VOCs, 0.50 mg/m³; formaldehyde, 0.05 ppm; total aldehydes, 0.1 ppm; and respirable particles, 0.05 mg/m³.

Counter tops and case goods are preferably made of low emission material such as the acceptable particle board products set forth above and should also utilize water based adhesives to bond pieces of the particle board together. Mechanical means for joining together the pieces of particle board, such as nails, wood screws, etc., are preferred so that the amount of adhesive can be kept to a minimum to minimize the emission load in the interior space. Wherever particleboard or composite wood surfaces are used in the interior space, it is preferred that they be sealed with a low emission acrylic sealants to prevent off gassing from the adhesives contained therein.

As wood solids typically have lower emissions than particle board products, they are generally preferred for all applications in which their greater cost can be justified by the property owner. Preferred wood species include basswood and birch since they tend to be better tolerated by the chemically sensitive than pine woods. It is preferred that cabinet front surfaces should include solid wood fronts and utilize low off gassing finishes like water dispersible urethanes, such as, Fabulon Crystal II, satin finish, and the water soluble sealers in the AFM Safecoat™ line. Similarly, wood solids are the preferred material for doors, trim and furniture of the interior space. Alternately, baked on finishes or baked on chemically inert laminate may be used, such as baked on acrylics. These finishes will typically have a lower VOC off gassing than traditional wood finishes. It is preferred that all wood finishes chosen meet or exceed the emission standards set forth herein for construction adhesives.

Counter tops are preferably granite, marble or stone, or other chemically inert natural products. Where cost is an issue, ceramic tile or high temperature wood laminate may be used, but are not preferred due to greater off gassing potential. Regardless of the materials chosen for the cabinets and countertops, they should meet the following standards: total VOCs, 0.25 mg/m³; formaldehyde, 0.025 ppm; total aldehydes, 0.05 ppm; and 4-phenylcyclohexene, 0.00325 mg/m³.

Bath tubs and the kitchen sink are preferably made of steel. The kitchen sink is preferably stainless steel, while the preferred bathtub is enameled steel. Plastic sinks and tubs should generally be avoided, where possible, to avoid off gassing issues. Bathroom fixtures are preferably ceramic or may also be stainless steel or enameled steel. Plastic plumbing pipes, fixtures and parts are generally to be avoided where possible to limit off gassing and potential for leaks which can lead to mold growth. However, where cost is an issue, low emission plastic plumbing pipes and fixtures may be used provided that they meet the standards set forth herein for construction adhesives. Low emission plumbing adhesives for use with plastic piping are available from North America Adhesive. Where the preferred metallic plumbing pipes are used, they should be properly insulated to prevent pipe sweating due to condensation which can lead to hidden mold growth. A low odor, silicone caulk may be used to seal the bathtubs and bathroom fixtures, suitable caulks include GE Silicone II™ and CSL Silicone 166/343™ from Webco Sealants. It is preferred that any caulk used in the interior space meet the requirements for emissions set forth herein for construction adhesives. The preferred low emission caulk products are also manufactured by AFM, Inc. under the trademark SAFECOAT. If the interior space includes a refrigerator, the water produced during defrosting is drained directly to a sink rather than into an evaporator tray under the refrigerator, as is common with many conventional designs.

All linens, draperies, bedspreads and soft seating should preferably be made from cotton. It is also preferred that mattresses contain 100 percent organic wool as fireproofing. Mattresses, covers and other any of the bed linens should be toxic chemical free with no bleaches or dyes used in their production. Upholstery fabric is preferably made from cotton and rayon fabrics which lack soil or stain repellants. Furniture stuffing and mattress padding is preferably made from untreated cotton felt, although other low emission padding materials may be used. Alternately, sealing covers sold under the mark Allertech® by Allergy and Asthma Technology Limited may be utilized to completely seal old pads or mattresses. In which case, the previously used pad or mattress may be reused. The mattress may also contain organic wool as fireproofing. Regardless of the textile materials or padding materials chosen for the interior space, they should meet the following standards: total VOCs, 0.50 mg/m³; formaldehyde, 0.05 ppm; total aldehydes, 0.1 ppm; and 4-phenylcyclohexene, 0.0065 mg/m³.

Turning to the selection of the furniture for the interior space, the materials selected should have the following characteristics. The materials should have low VOC off gassing rates, lack chemical irritants and common allergens, facilitate thorough cleaning, and be unfavorable to the growth of biological agents which commonly contribute allergens, toxins or other undesirable bio-aerosols. For these reasons, the following criteria should be followed in selecting furniture for the indoor space. First, materials such as conventional fiber board which off gas significant quantities of noxious volatile organic compounds should be avoided. The same pressed wood products described above for fiber board substitute materials regarding cabinetry may be used as a substitute for conventional fiber board. Depending upon the type of fiber board used, it may be beneficial to use a low emission acrylic sealant or a high temperature laminate finish to seal the exterior surface of the fiber board to prevent off gassing to the interior space. The AFM SafeCoat™ product line further includes acceptable water soluble wood sealant products. As discussed above, the use of wood solids are preferred due to their low chemical emission characteristics. However, it is recognized that cost considerations generally militate against the use of woods solids. Because wood solids tend to have lower chemical emissions in many cases, it is not necessary to completely seal their surfaces; however, it is preferred. Thus, acceptable finishes include low emission, water based polyurethane coatings which provide moisture resistance and assist clean up of the furniture, even though some of those materials may not completely seal the wood solids. High temperature laminate finishes are also an acceptable alternative. The use of volatile wood stains should be generally avoided due to emission concerns; acceptable chemically inert, low emission wood stains include those available in the Safecoat™ line. Further, each of the adhesives used in the fabrication of the furniture must be analyzed for VOC off gassing since many of the conventional furniture adhesives have unacceptably high off gassing rates. Acceptable furniture adhesives are available in the Solvent Free™ line manufactured by North America Adhesives. Whenever possible, it is preferred that furniture components are joined together primarily by mechanical means such as nailing, wood screws, tacking or staples to avoid the use of adhesives the minimize the total chemical load the interior space. Generally, where adhesives are necessary, they should be low emission adhesives that meet or exceed the emission standards previously set forth above for adhesives. Where the property owner wishes to reuse existing furniture that has exhibited unacceptable chemical pollutant or allergen levels, the wood surfaces can be sealed with a low emission wood sealant and, where necessary, padding and textiles can be removed and replaced with low emission padding and textiles.

The textiles chosen for use in the furniture are preferably untreated cotton lacking flame retardants, stain repellants, chemicals dyes, and/or chemical bleaches. Where dyes are to be used for coloring fabric used within the space, organic dyes are preferred and each of the dyes to be used should be analyzed for both short term and long term VOC off gassing. Also, low emission furniture padding is preferred. The low emission textiles and padding should meet or exceed the emission standards set forth above for those materials. Further along these lines, it is greatly preferred that the furniture used in the indoor space is custom designed to meet the specifications set forth herein. This is preferred since the vast majority of conventional furniture will not meet the guideline set forth herein for low chemical emissions and avoidance of potential chemical irritants. Regardless of the type of furniture chosen for the interior space, each piece of furniture should meet, at a minimum, the following standards: total VOCs, 0.250 mg/m³; formaldehyde, 0.025 ppm; total aldehydes, 0.05 ppm; and 4-phenylcyclohexene, 0.00325 mg/m³.

As discussed above, it is preferred that the walls be painted with low emission paint; however, acceptable wall coverings such as low emission wallpaper may also be used. The wall paper should be adhered to the wall using low emission adhesives that meet or exceed the standards discussed above. Low emission wallpaper sold under the trademark Earth Friendly™ is available from New Moment Environmental Contract Wall Coverings. Regardless of the wall covering chosen for the interior space, it should meet the following standards: total VOCs, 0.50 mg/m³; formaldehyde, 0.05 ppm; total aldehydes, 0.1 ppm; and 4-phenylcyclohexene, 0.0065 mg/m³. These standards include the contribution to the chemical load provided by any adhesive used to affix the wall paper to the wall as well as to the wall paper itself.

The preferred heating system is a hyrdronic radiant floor heating unit. Such systems include tubing that is installed in the flooring below the surface flooring material. As mentioned above briefly, when using such a radiant floor heating system, it is preferable that wall to wall carpeting be avoided. As a result, either hardwood flooring or tile flooring is preferred with the radiant floor heating systems of this invention. One advantage of radiant floor heating system is that a more efficient ventilation system can be designed for maintaining indoor air quality without the compromises which would be necessary if the ventilation system also included that heating and air conditioning components. As seen in FIG. 1, the heating system 50 further includes the following components an electronic hot water heater 52 coupled by piping 54 with a pump 56 which circulates the heated water to a heat exchanger 58. This unit exchanges heat out or into a glycol loop 60 which is associated with a second pump 62 that circulates the glycol base heated media throughout the floor piping 64 of the interior space. Optionally, the water heater may also be designed to provide hot water to the space as is shown in FIG. 1.

For the cooling exterior air circulated into the space in the summer months, a small air conditioning unit includes a condensing coil associated with the ventilation unit. In those months, air is cooled by the cooling coil and then reheated in the second stage heat exchanger. The net effect is primarily dehumidification, rather than cooling of the exterior air brought into the ventilation system. Additional cooling is provided by removing heat from the space via the radiant floor piping 64 so that the warmed glycol exiting the space exchanges heat at the heat exchange unit 58. Heat is withdrawn from the warmed glycol and vented to the exterior of the building with the cooled glycol returned to the radiant floor piping 64.

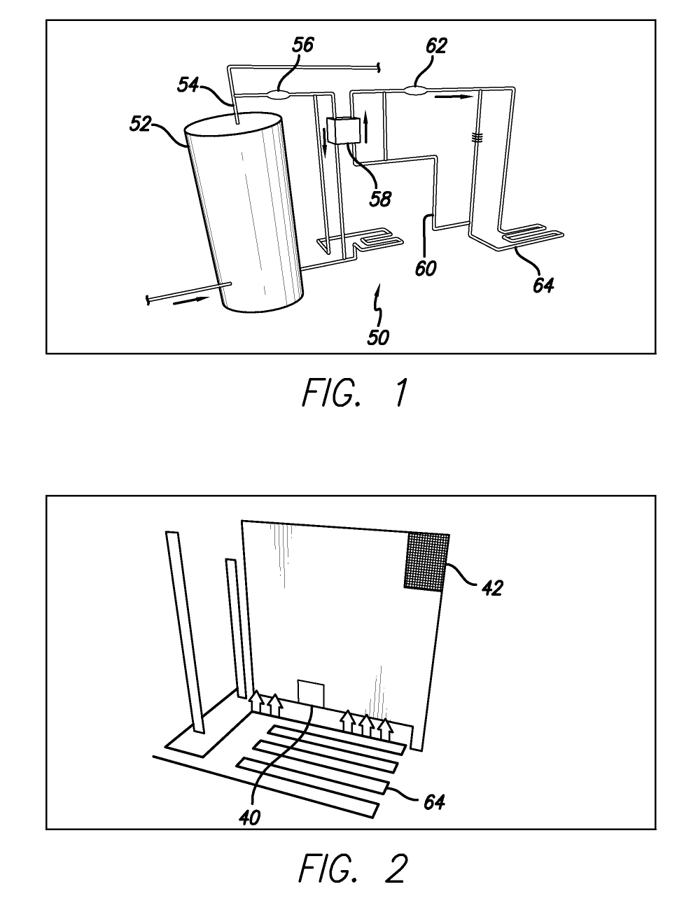

The ventilation unit 21 employs a method of ventilation known as displacement or stratified ventilation which is illustrated in FIG. 2. Air ducts 40 for the ventilation system are located near floor level, and the air circulated there through is provided at a relatively low velocity to avoid causing drafts in the interior space. This arrangement minimizes the amount of high velocity air circulation in the space so that particulates are not blown about it by excessive air velocity as is common in some systems in which heating, ventilation and air conditioning are combined in one system (“HVAC,” systems). However, at the same time, the amount of air circulation in this embodiment of the invention is sufficient to permit removal of stale, polluted or contaminated air from the interior space, the preferred air change per hour rate (ACH) for exchange outdoor air concentration of 0.8. In many existing spaces where cost is an issue, the existing HVAC system will be reused after proper cleaning and modification to add HEPA filtration, activated charcoal filtration, and/or one or more of the following purification sub-systems ion exchange, UV light, or ozone generation units. In the hospitality industry, many hotel room spaces do not have air exchange systems to bring in outside air. In these cases, it is preferred that the HEPA filters and activated charcoal filter or other air purification the subsystem(s) are utilized and maintained since the air has very limited outdoor exchange under normal circumstances. Further, it is preferred that the air turnover within such rooms be somewhat higher in order to ensure that filtration system can remove indoor generated pollutants from the air since “fresh” outdoor air will not be routinely exchanged into the space. Under these circumstances, it is preferred that the air handling system has an interior air recirculation rate which will draw all the air in the space through the system about eight cycles per hour.

Returning to the system of FIG. 2 as mentioned above, the duct 40 for the air re-circulated into the room is near floor level and the return 42 is located near the ceiling. This design is efficient for improving indoor air quality because pollutant sources typically emit gasses that are (a) warmer than the surrounding ambient air and/or (b) of lower density than the ambient air. This causes those gasses to rise near the ceiling of the interior space where the air returns 42 are located. With the preferred low velocity system of the present invention, it is possible to have a heating, air conditioning system and ventilation system which minimizes stirring particulates within the space, but at the same time is efficient in removing pollutants from it.

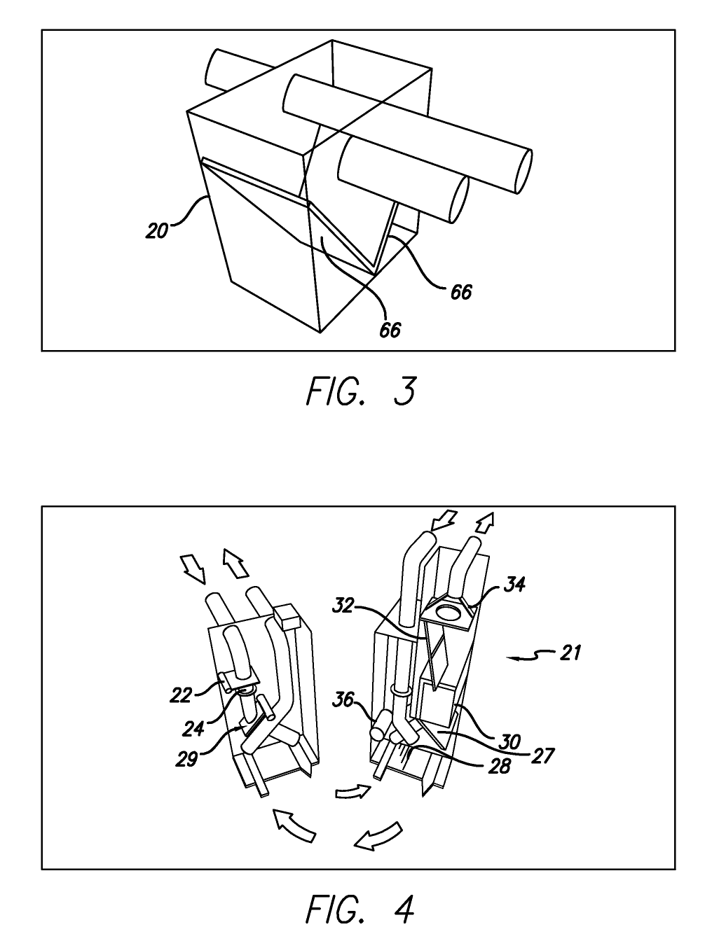

As best seen in FIGS. 2-4, the ventilation unit 21 includes an air purification system. The air purification system may include three filters, the first; a washable “rock catching” filter made of coarse aluminum mesh is located outside the house in the intake exhaust unit 20. The second filter 30, a pleated paper filter, takes out particles of a size of down to 0.03 microns with 99.97 arrestance. This second filter 30 is a HEPA filter and is located within the main ventilation unit. A third filter, an activated charcoal filter, provides for removal of organic compounds and odors. The activated charcoal filter 32 provides significantly more resistance than conventional filtration and requires additional fan power to maintain sufficient airflow, even at the lower velocities which are preferred in this embodiment of the invention. Optionally, if desired, the charcoal filtration portion of the system may be designed to be turned off and on as is necessary when the owners detects an odor or suspects off gassing is present. Under those circumstances, the filtration unit containing the charcoal filter should include an auxiliary fan 34 which is wired to a manually controllable switch (not shown) for manual activation by the occupant. However, in many situations it may be preferred that the activated charcoal filter is constantly operating, in which case, both the switch of the auxiliary fan would be unnecessary. In this embodiment of the invention, a relatively larger capacity fan may be necessary to provide the relatively high air pressure required to pass air efficiently through an activated charcoal filter.

Referring, in more detail, to the one ventilation unit of FIG. 2, it includes an air intake 20, damper 22, intake fan 24, first heat exchanger 26 and ultra-violet light source for irradiating the intake air and killing off any biological contaminants. The ventilation unit 21 is also provided with a heating/cooling coil 28 which can provide heat to the intake air during the winter months or cool the intake air during the summer months. The air will then pass through a second intake heat exchanger 29 through the second level particulate filter 30 and onto the third level charcoal filter 32 at which point it may be accelerated by an auxiliary fan if it is activated. The air is then exited to the ducts which lead to the air duct 40 for the indoor space. When the contaminated air is removed from the interior space via the air return 42, it passes into the duct work to the ventilation unit 21 where it is accelerated by an exhaust fan 36 and passes through first and second heat exchangers (27, 29) and is exhausted to the out-door environment.

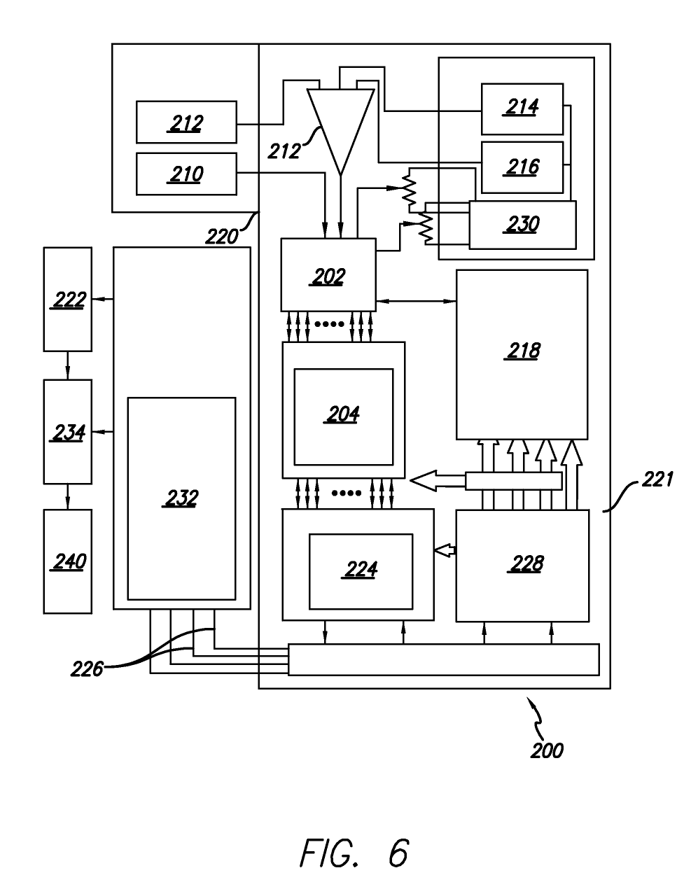

In one preferred embodiment of the invention in which individual room air handling is contemplated, such as commonly used in the hospitality industry, commercially available air purification systems which include both a sanitizing mode and purification mode are utilized. One preferred air purification system is the Fresh Air model by Ecoquest International of Greenville Tenn. This room sized unit includes positive and negative ion pulse generators, dual output ozone generator, UV light, and lint screen (conventional dust filter), and may be optionally fit with a HEPA type filter. Preferably, such a room sized air purification unit is operably coupled to and air monitoring system, if available. The coupling of the air purification system 240 (see FIG. 6) of to the air monitoring system 120 or sensor array 200 may be a hard wire connection or a wireless connection, such as, UV light or RF systems.

The design planning stage can optionally be conducted after an existing interior space has been physically inspected and samples have been analyzed for problematic conditions. The samples to be collected preferably include both air samples and surface particulate samples, but may include only air samples, if desired. The samples are analyzed for the following:
Particulates
Volatile Organic Compounds
Mold
Other common allergens
Other known or potential chemical carcinogens or irritants

This testing for mold, particulates, allergens and chemical compounds and other potential chemical irritants provides a snap shot of the indoor air quality for allergens, bio-aerosols, VOCs and other chemicals of concern within the pre-existing space. It is also beneficial to have similar testing on air sample from the outdoor environmental surrounding the interior space. Comparisons of the indoor and outdoor air quality are beneficial in helping to identify whether sources of indoor air contaminants or pollutants have originated from an indoor source or from the outdoor environment. This is particularly true for mold testing since outdoor mold counts can vary significantly between seasons and during local whether events. The preferred mold counts comparisons are performed using a protocol established by Environmental Microbiology Laboratory, Inc. of San Bruno, Calif. and are identified by the service mark MOLDSTAT. Comparison of indoor and outdoor mold counts provides a scientific method to assess whether the indoor space in question contains more of a certain organisms than should normally be present. The preferred sampling protocol includes comparison of volumes of measured sampled air and results are expressed in terms of volumetric measurements. These testing measurements also provide a standard to assess the improvements in indoor air quality after the space after has been reconstructed using the allergy friendly methods of the invention.

The goal of the biological sampling is to help determine whether the biological particles present in a particular environment may affect or causing irritation in certain individuals. Sampling is also used to locate the sources of indoor microorganisms and facilitate an effective remediation. Some bacteria and fungal spores can cause disease only when they are alive (viable), while others are capable of producing allergies or irritation even when no longer living. Live culture testing may permit greater accuracy in speciating some fungal organisms present. However, spores vary widely in their ability to grow and compete on laboratory media. This may result in an inaccurate characterization of the area sampled. Therefore, a complete sampling protocol for the biological flora in any environment should preferably use both a culturable and non-culturable sampling method. When time and budget constraints prevent such full scale testing, non-culturable spore trap sample is usually the best choice when only one sampling method can be used.

Non-culturable spore trap samplers draw measured volumes of air through the sampling device for a specified length of time. The collection surface is a coated glass slide. Particles in the air (spores, dust, etc.) impact onto the sticky surface and are “trapped” for later analysis. The preferred spore trap is an Air-O-Cell™ Cassettes manufactured by Zefon Analytical Accessories. The primary advantage of Zefon's Air-O-Cell is their relatively low cost and small size (easy to transport, useful in small spaces). Allergenco/Blewstone Press and Burkard Manufacturing both make spore trap sampling devices which accept standard glass slides. All of these devices have excellent aerodynamic characteristics and are very effective in monitoring airborne particles and organisms.

Effective interpretation of results is based on the comparison of indoor and outdoor samples. There are currently no government guidelines or regulations to indicate “safe” or “normal” mold spore levels, however, typically indoor counts (from conventional rooms) are about 40 to 80 percent of outdoor spore counts, with the same general distribution of spore types present. Variation is an inherent part of biological air sampling. Thus, the presence or absence of a few genera in small numbers should not be considered abnormal. However, large counts of certain mold species or of certain genera, e.g. Stachybotrys, are cause for concern and require immediate remediation. Mold species, such as those within the Stachybotrys genus, are of particular concern because they have been reported to contribute air born mycotoxins to indoor environments. With the methods of the invention, it is expected that the mold counts will be less than about 10 percent of the outdoor mold spore count. As mentioned above, it is preferred that testing for bio-aerosols be undertaken if very high mold spore count for any mold species are found, or if lower spore counts for certain problematic species of the genus Stachybotrys or Aspergillus are found. In that case, testing according to one or more of the following protocols may be undertaken to ensure that any source for the generation of problematic bio-aerosols is appropriately remediated.

Traditional bio-aerosol analysis methods involve time-consuming particle collection and laboratory analysis of live culture samples. The colonies are typically allowed to grow on culture media for between five and seven days. The colonies are then counted and typically identified by trained microbiologists using stereomicroscopes as well as microscopes. The identification process may involve characterization of the taxonomy of the colonies and the individual fungal cells and spores. Typically, a calculation of colony forming units (CFU) per volumetric air sample is made. Genus level identification of most species is adequate for designing an appropriate remediation strategy. With species of the genus Aspergillus, the identification process is taken to the species level because only certain species of that genus are known to produce problematic mycotoxins. Where problematic mycotoxins are believed to be present, additional remediation steps may be necessary, such as, filtering the air within the sealed remediation zone when the work is being performed.

An alternate protocol for bio-aerosol analysis involves ionizing the bio-aerosols in a volumetric air sample and analyzing the ionized materials in a mass spectrometer. The mass spectrometer detects single particles in a known air volume and tallies the number and types of bio-aerosol particles by identifying chemical that are associated with certain genera and species of microbes, pollen, or insect parts to obtain the total bio-aerosol concentration. Such mass spectrometer analysis protocols typically involve an estimated CFU count for mold genera and species which is base on a calibration of the mass spectrometer readings to live cultured colony analyses. Currently, collected air samples must be transported to a laboratory for the mass spectrometry analysis due to the bulk of the equipment. A variety of portable analyzers are currently in development. Upon successful completion of their development, the use of such portable bio-aerosol mass spectrometers which could detect, ionize aerosol particles, and identify genus and species of the bio-aerosols as well as providing CFU counts in real time would be preferred.

The chemical air sampling for the interior space is preferably performed pursuant to a variation of ASTM standards D-5116-97 and D-6670-01. The indoor space is allowed to equilibrate using the current air circulation and/or HVAC system. It is envisioned that the most common indoor spaces will typically be on the order of the 32 m³ room of the standard so the acceptable concentrations for most indoor pollutants will be about the same as those set forth above. Preferably, the testing includes quantifying at least total VOCs, formaldehyde, total aldehydes, 4-phenylcyclohexene, styrene, perchloroethylene, parardichlorobenzene, alkylphenols, ethoxylates and dibutyl phthalates. If it is known or suspected that other carcinogens and reproductive toxins have previously been used in constructing the interior space, have been used in maintaining it, or were formerly present, testing should also be conducted for those compounds. List of known and suspected carcinogens and reproductive toxins can be found in California Proposition 65, the U.S. National Toxicology Program (NTP), and the International Agency on Research on Cancer (IARC). Further, if it known or suspected that the outdoor environment in the vicinity of the indoor space contains U.S. EPA regulated primary or secondary outdoor air pollutants, it is preferred that testing of the interior space for those pollutants should also be conducted. Unless explicitly stated, the measurements for these chemical compounds are via this same preferred protocol described above.

Where allowable emission levels exceed the maximums allowable under the state of Washington's indoor air quality program for new construction, the US Environmental Protection Agency's procurements specifications, the recommendations from the World Health Organization, and/or Germany's Blue Angel Program for electronic equipment, all materials that are believed to have contributed to those reading should be remediated. When multiple emission maximum values are recommended by these authorities, it is preferred that the lesser or more stringent level is used as the acceptable emission value.

The next step is to remediate the interior space to rid it of sources of common allergens, materials harboring or likely to harbor mold or other organisms (such as insects) which commonly generate bio-aerosols, VOCs or other chemical irritants found in the analysis. Typically, this remediation process will include removing all furnishings, carpeting (or other problematic flooring material), wall coverings and window treatments. After those materials are removed, a visual inspection of all walls, floors, ceilings, duct work, as well as heating, ventilation, and air conditions systems is conducted. Special attention is paid to the bathroom or other areas which include water pipes and fixtures since leaks, condensation on pipes, or excessive humidity in shower areas can provide moisture which facilitates growth of mold in the interior space. Any materials showing mold growth, such as dry wall, framing material, plywood, cabinetry is removed from the space.

After the inspection is complete, a plan for repairs of to re-mediate any issues turned up during the inspection is formulated. If mold growth is one of the issues identified, then the preferred remediation techniques will include sealing the interior space and providing appropriate exterior ventilation to prevent the spread of mold spores within the interior space or to other rooms or compartments within a building. Furthermore, any dry wall, pressed wood, wood products or other materials which show mold growth should be completely removed from the space, rather than being surface treated with a fungicide. Removal is preferred because even the dead mold may give off mycotoxins and allergens. However, if structural wood materials such as wall studs, floor joists, or ceiling joists, contain surface mold growth, it may be ground out of the structural member, treated with a fungicide, and then sealed with a low VOC sealant. Of course, if the amount of fungal growth is so extensive that the structural integrity of structural member could be comprised, the structural member should be replaced. As mentioned above, effective remediation of mattresses or cushions may be effected by utilizing sealing covers.

The next step is to begin to reconstruct the interior space. All materials and products shipped to the site are preferably inspected and approved for conformance to the products specifications. As set forth above, all of those materials have been approved for use in the space based on the specifications of the manufacturer that has been previously reviewed by appropriate personnel, such as a CIH. At this stage of the reconstruction of the space, it is preferred that selected samples of the delivered materials or products are periodically tested for off gassing of VOCs, the presence of other common chemical irritants, suspected bio-active compounds, and potential carcinogens. Such testing should be conducted according to the protocols set forth above. Non-conforming materials are rejected and replaced with conforming materials. The inspection and approval process includes not only building materials, but also wall coverings, window treatments, flooring coverings, and furniture.

During construction best cleaning practices of the site must be adhered to so that dust, dirt, allergens, construction debris are not trapped behind walls, under flooring material or within any other structure of the interior space. Those cleaning practices include cleaning all newly installed and existing duct work within the space. Particular care should be taken to remove “wood dust,” more commonly known as “saw dust,” since it has been recently listed in the Tenth Version of the NTP as a potential carcinogen. Upon completion of construction, the room is thoroughly cleaned with all surfaces wiped down, and with the floors being mopped and then, after drying, cleaned with a vacuum cleaner equipped with a HEPA filter to remove particulates.

If an existing HVAC system is to be reused, it is strongly preferred that HEPA air filtration and activated charcoal filtration capabilities are added to the existing system. Further, if the system has an outdoor air exchanger, it is preferred that the indoor/outdoor exchange rate be optimized based on samplings from the indoor and outdoor air quality. In most environments where the outdoor air is of relatively high quality, an air exchange rate of 0.8 ACH is generally preferred. However, where the outdoor air is of low quality, lower exchange rates are preferred.

Upon completion of the initial cleanup, the air purification system is activated and the indoor air quality room in the interior space is allowed to stabilize, which will typically take at least twenty four hours. This can be done in conjunction with a HVAC system or as a stand alone air purification unit as described above. Air samples and preferably surface samples are taken for mold, particulates, allergens, total VOCs and other problematic chemical compounds to provide a baseline of the those compound in the completed allergen friendly interior space. Further, testing for bio-aerosols may be undertaken and a baseline set where circumstances warrant this optional procedure. As mentioned above, it is also beneficial to have similar tests performed on air sample from the outdoor environmental surrounding the interior space. Comparisons of the indoor and outdoor air quality data as well as comparisons between the pre-remodeling space data and the post allergy friendly remodeling data provide the best indication of the beneficial effects of the methods of the invention. Further, comparisons with similarly situated conventional, interior spaces within the same building are also beneficial to demonstrate the efficacy of the present methods. This is particularly true for mold testing since outdoor mold counts can vary significantly between seasons and during local whether events.

An important step in the process of the invention is to train housekeeping and maintenance staff in correct cleaning and maintenance procedures for the interior space. Such training includes the use of approved cleaning agents and cleaning methods for the interior space. This training includes training the laundry staff to use only the approved detergents and the avoidance of conventional chemical bleaches and fabric softening products. Acceptable detergent, bleach, and fabric softeners products are available from Allergy and Asthma Technology Limited under the trademark AllerTech™. The cleaning agents should generally include only cleaning solvents, detergents and other products that off gas VOCs at very low levels and do not contain the problematic chemical compounds or chemical irritants. Examples of acceptable low emissions solvents, cleaning supplies and detergents are available from Allergy and Asthma Technology Limited under the trademark AllerTech™. Regardless of the low emission cleaning agents chosen, they should meet the following standards: total VOCs, 0.50 mg/m³; formaldehyde, 0.05 ppm; and total aldehydes, 0.1 ppm. Further, the HEPA vacuum cleaner should be used to clean carpets, tile and hardwood floors. The HEPA vacuum should preferably reduce dust mite, mold spore, and dust particle contents by at least ninety five percent. After cleaning procedures are complete and the room has stabilized, expected to be at least several hours, the count for total respirable particles in the interior space should be no higher than 0.1 ppm.

Further important steps in the methods of the invention is the training of the maintenance or building engineering staff to maintain each of the filters in the air purification system and/or HVAC systems including any first level filter, HEPA filter, activated charcoal filter, and any HEPA vacuum cleaner filter. Unless these filters are adequately cleaned and/or replaced in accordance with the filter manufacturer's specifications, these filtration systems will not operate efficiently, and in extreme cases of neglect, may actually contribute to diminution of indoor air quality. Any cleaning products utilized for the filters must also be low VOC off gassing products which lack any common chemical irritants.

In one preferred aspect of the methods of the invention, approval labels are attached to each of the interior furnishings, window treatments, carpets, etc. which identify the material or item as being allergy friendly. An inventory of the approved interior furnishings, window treatments, carpets, etc. is taken to make accurate comparisons with the contents or the interior space over long periods of time. In this way, a simple visual inspection of the interior space, the inventory, and the approval tags can reveal if any non-conforming or unapproved furnishings, carpets, window treatments, etc. have been introduced into the space. Further, if additional materials or items have been added to the interior space, they should also be tested and bear the approval label when passing the standard set forth herein.

In another preferred embodiment of the invention, a real-time air quality monitoring system is installed and utilized within interior space. The term “real-time” as used herein refers to monitoring the equipment that either continuously monitors air quality or performs the monitoring testing processes at least on one occasion per per a relatively short period of time, for example, once per ever a five minutes. Optimally, it is preferred that air quality is monitored at least several times per minute. The purpose of the system is to detect any significant variations from acceptable base line levels for major indoor contaminants in a timeframe which is short enough that active measures can be taken to discover the source of the problem and remediate it.

Such a monitoring system should preferably include at least sensors for temperature, humidity, carbon dioxide concentration, carbon monoxide concentration, and a broad spectrum VOC sensor. A suitable sensor array for use within such a system is sold under the trademark the “Nose” by PureChoice, Inc. of Lakeville, Minn. The system may include multiple communication networks, such as, those described in detail in U.S. Pat. No. 6,782,351 issued to Pure Choice. The advantages of utilizing a multiple communication network such as that set forth in the '351 patent is that an off-site, expert service can be responsible for analyzing the relatively complex data collected by the sensors system and identifying potential problems. Alternately, the air quality sensor may be coupled directly to a microprocessor and a data storage device with the processing of the air quality data, its storage and archiving accomplished by a single computer or single, private computer network. If the system includes multiple air quality sensor arrays, and is preferred that the sensor arrays be coupled to a network router which is then link to multiple communication networks, a single computer, or single private computer network.

The simpler single computer and single computer network system configurations are advantageous in situations where a relatively sophisticated property owner has the resources necessary to manage and analyze the complex air quality data that can be generated by multiple sensor arrays. Archiving and data processing systems at either an on-site or a remote data collection site should include a controller programmed to automatically acquire over the network the air quality data from one or more sensor array assemblies and to automatically store air quality data in a database. In situations in which multiple heating, air purification and/or air-conditioning units are utilized within a building or site, the microprocessor can be coupled to one or more, and preferably each of, the multiple heating, air purification and/or conditioning and units.

In an embodiment of the invention which is particularly suited to the hospitality industry, the real-time air quality monitoring system is operably connected to the air purification system. Optimally, the air purification system includes multiple modes of operation which vary in their efficiency in removing particular sets of airborne contaminants. For example, ozone generators which include a high also output sanitizing mode are particularly efficient for neutralizing strong orders, as well as potentially toxic or pathogenic airborne biological agents, such as, e.g., bacteria, mold, mycotoxins or viruses. However, the generation of levels of ozone which are most effective at such neutralization of biological agents can be sufficiently high to cause a slightly unpleasant “ozone” odor. Thus, linking the air quality monitoring system to the air purification system provides a means to selectively activate the ozone sanitizing mode when this system detects that no human occupants are within interior space. One way in which this can be accomplished is to program in the microprocessor to recognize pre-determine lower levels of carbon dioxide which are typically associated with the absence human respiration in an interior space. Once detected levels of carbon dioxide drop below the predetermined lower level, the microprocessor is programmed to recognize the event and sends an electrical signal to the air purification system to activate its ozone sanitizing mode. When the detected carbon dioxide levels rise again to above a predetermined level, the ozone generator may be switched to its lower ozone output sanitizing mode or it may be deactivated. Of course, other air quality parameters may be utilized to actuate a switch in air purification modes. For example, levels of nitrous oxide and/or carbon monoxide may also be used to activate the air sanitizing mode of the ozone generator when an interior space is believed to be unoccupied human.

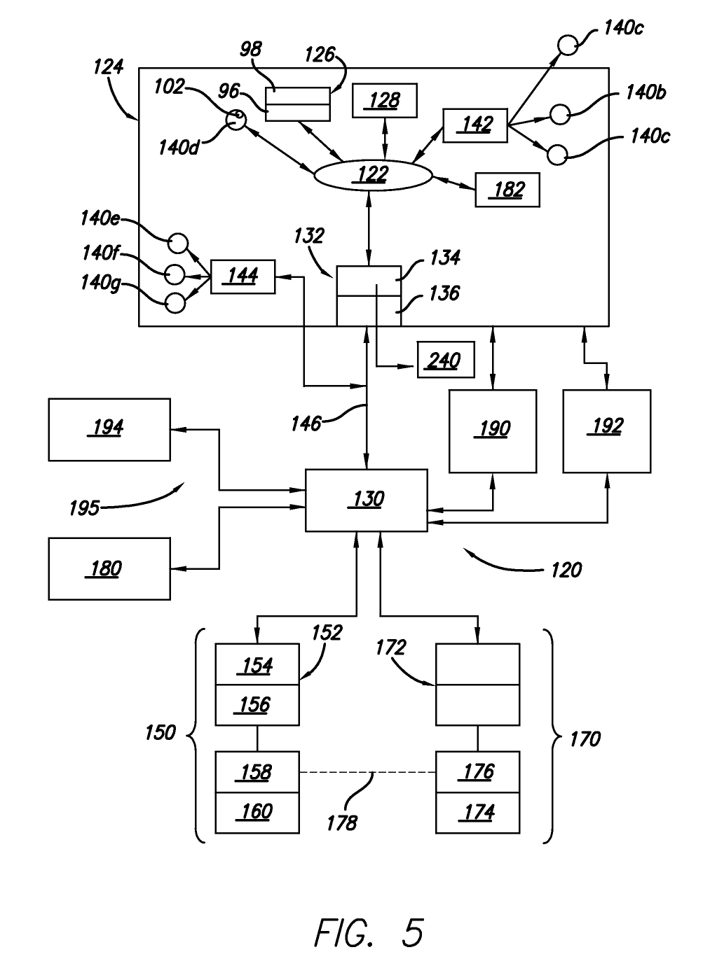

FIG. 5 is a schematic illustration of such a preferred air quality monitoring system 120 coupled to a private communications network 122 at site 124. The private communications system 122 includes network infrastructure 123, such as wiring and access ports (e.g., RJ-45, RJ-14, RJ-11, and fiber optic jacks), located around the site 124. However, it is contemplated that the access ports may be connected to the private communications network 122 by one or more RF communications devices (not shown). The private communications network 122 can be a local area network (LAN) (e.g., as an Ethernet LAN or a token ring LAN), an Intranet, an Extranet, or a Virtual Private Network (VPN). In the illustrated embodiment, private communications network 122 is a LAN with computer workstations 126, 128. As used herein, “private communications system” means a network, such as a local area network, an Intranet, an Extranet, a Virtual Private Network or any other communication structure designed to carry data between one or more computers located at a site. The private communications system is typically digital, but may contain one or more analog segments, such as a modem, in the various communications channels.

Various embodiments of private communications systems suitable for use in the present invention are disclosed in U.S. Pat. No. 5,802,285 (Hirviniemi); U.S. Pat. No. 5,978,373 (Hoff et al.); U.S. Pat. No. 6,157,950 (Krishnan); U.S. Pat. No. 6,188,691 (Barkai et al.); and U.S. Pat. No. 6,215,789 (Keenan et al.).

The illustrated private communications network 122 communicates with public communications network 130 through gateway 132 and communications channel 146. The gateway 132 typically includes a web server 134 and a proxy server 136. The proxy server 136 is a server that acts as an intermediary between the private communications network 122 and the public communications network 130 so that the site 124 can ensure security and administrative control.

The proxy server 136 may also be associated with or part of firewall server that protects the private communications network 122 from outside intrusion. A firewall is a set of related programs, located at the gateway 132 that protects the resources of a private communications network 122 from users from other networks. Where private communications network 122 is an Intranet, a firewall prevents unauthorized users from accessing the network 22 and controls what outside resources users of the private communications network 212 have access to. A firewall, working closely with a router program, examines each network packet to determine whether to forward it toward its destination. A firewall also includes or works with a proxy server that makes network requests on behalf of workstation users.

Various techniques for passing data between a private communications network and a public communications network are disclosed in U.S. Pat. No. 5,944,823 (Jade et al.); U.S. Pat. No. 5,963,146 (Johnson et al.); U.S. Pat. No. 5,999,973 (Glitho et al.); U.S. Pat. No. 6,122,281 (Donovan et al.); U.S. Pat. No. 6,181,681 (Hiscock et al.); U.S. Pat. No. 6,172,616 (Johnson et al.); and U.S. Pat. No. 6,205,490 (Karapetkov et al.).

The public communications network 130 can be a wide area network, an Extranet or the Internet. The Internet, sometimes called simply “the Net,” is a worldwide system of computer networks—a network of networks in which users at any one computer can, if they have permission, get information from any other computer (and sometimes talk directly to users at other computers). Physically, the Internet uses a portion of the total resources of the currently existing public telecommunication networks. Technically, what distinguishes the Internet is its use of a set of protocols called TCP/IP (Transmission Control Protocol/Internet Protocol). Intranets and Extranets also make use of the TCP/IP protocol. Suitable WANs for use with a system of invention may be any connection, such as a telephone line, X.25 line, lease line, asynchronous link, SNA network, integrated services digital network (ISDN).

A plurality of sensor assemblies 140a, 140b, 140c, 140d, 140e, 140f, 140g (collectively referred to herein as “140”) are coupled to the network infrastructure 123 of the private communications network 122. In one embodiment, one or more sensor assemblies 140 are coupled directly to an access ports (e.g., RJ-45, RJ-14 and RJ-11 jacks) on the site 124, such as for example the sensor assembly 140d that is compatible with Ethernet protocol. Consequently, the sensor assemblies 140 can typically be located throughout the site 122 without the need for additional wiring. As used herein, “coupled” refers to an interconnection that permits data to be exchanged between two or more device. The sensor assemblies 140 preferably permit seamless device plug-in. This feature gives users the ability to plug the sensor assembly 140 into the private communications network 122 and have the network 122 recognize that the sensor assembly is there and applies the appropriate drivers. The user doesn't have to tell the network 122.

The sensor assemblies 140 are preferably positioned at various distributed locations in a particular site 124. The number and arrangement of the sensor assemblies 140 shown in FIG. 1 is for illustrative purposes only and can vary depending upon the air quality monitoring requirements. As will be discussed below, each of the sensor assemblies 140 includes one or more sensors adapted to measure a level of an air quality attribute (see e.g., FIG. 2). As used herein, “air quality attribute” refers to a characteristic of the ambient air including without limitation temperature, humidity, pressure, the level of a particular gas or chemical (such as VOCs), or particulate, such as mold, toxins, dust, and the like.

The sensor assemblies 140 can be coupled to the private communications network 122 using a variety of configurations. In the illustrated embodiment, the sensor assemblies 140a, 140b, 140c are coupled to a communications interface 142, which is coupled to the private communications network 122. The communications interface 142 preferably converts sensor data from the sensor assemblies 140a, 140b, 140c into a protocol compatible with the private communications network 122. For example, the communications interface 42 can convert sensor data into an Internet protocol. In an alternate embodiment, the sensor data is converted to a first protocol at the sensor assembly 140 and the communications interface 142 converts the first protocol to a second protocol compatible with the private communications network 122. For example, the sensor data is converted at the sensor assemblies 140a, 140b, and 140c to an industrial control language sold under the trade name Lontalk™ available from Echelon Corp. Lontalk is a desirable format for the air quality data because of its compatibility with many existing heating, ventilating and air conditioning systems (HVAC). The communications interface 142 may, in turn, convert the air quality data to a format compatible with the private communications network 122, such as Internet protocol

In on aspect of the illustrated schematic of FIG. 5, sensor assembly 140c is located outside of the physical confines of the site 124. For example, the sensor assembly 140c can be located outside of the building defining the site 124 to measure air quality attributes that may affect air quality within the site 124. Sensor assembly 140c is useful to measure the migration of air quality attributes into and out of the site 124. Positioning one or more sensor assemblies outside of the site 124 is also useful for predicting trends in air quality attributes within the site 124 or for analyzing the efficiency of air purification measures within the site 124 relative to the air quality of the air outside of the site 124.

In another embodiment, the communications interface 142 can be provided with each sensor assembly 140 so that the sensor assembly 140 can be coupled directly to the private communications network 122, such as sensor assembly 140d is connected directly to the private communications network 122. In this embodiment, microprocessor 102 located within the sensor assembly 140 d converts the sensor data to a format compatible with the private communications network 122, such as, for example, an Ethernet protocol. In another embodiment, the sensor assemblies 140e, 140f, 140g are connected to a communications interface 144 that is coupled to the portion of the communications channel 146 located at the site 124 downstream of proxy server 136. This embodiment provides additional security for the private communications network 122.

The site 124 is preferably assigned a unique site identification number. Each sensor assembly 140 is preferably assigned an unique sensor assembly identification number. In another embodiment, the microprocessor 102 can assign an unique sensor identification number to the data stream generated by each sensor within the sensor assembly 140. In any of these embodiments, the air quality data can be correlated to a particular air quality attribute measured by a sensor assembly 140 at the site 124.

In one preferred embodiment of the invention, air quality data is uploaded from the sensor assemblies 140 to the private communications network 122 and subsequently through the public communications network 130 to a second private communications network 150. The second private communications network 150 is also referred to as the archiving and processing system. The second private communications network 150 includes a gateway 152, typically with a proxy server 154 and a web server 156 connected to a controller 158 that processes air quality data and maintains database 160. The second private communications network 150 is preferably located remotely from the site 124 to provide secure archiving. The second private communications network 150 also provides secure access to the air quality data through the public communications network 130 for both users at the site 124 and users at remote sites 180, 194.

A third private communications network 170 may optionally be used for redundancy. The third private communications network 170 also includes a gateway 172 and a controller 174 that maintains database 176. The third private communications network 170 is preferably located at a site physically remote from both the site 124 and the second private communications network 150. A synchronization connection 178 is optionally provided to synchronize the databases 160, 176.

Air quality data may be sampled and can be uploaded through public communications network 130 to one or more private communications networks 150, 170 either continuously or at discrete time intervals. In that case, it is preferred that the sensor assemblies 140 are programmed to sample air-quality on a continuous or near continuous basis. For example, if communications channel 146 is a dedicated communications line, a continuous stream of air quality data can be sent to the database 160 and/or 176. A dedicated line is a telecommunications path between two points that is available 24 hours a day for use by a designated user (individual or company). It is not shared in common among multiple users as dial-up lines are. A dedicated line can be a physical path owned by the user or rented from a telephone company, in which case it is called a leased line. A synonym is nonswitched line (as opposed to a switched or dial-up line).

In another embodiment, air quality data is uploaded to the private communications network 150 and/or 170 at discrete time intervals, such as every 10, 20 or 30 seconds, whether or not the channel 146 is a dedicated line. In such a case, it is preferred the defense or assemblies 140 are programmed to analyze air quality at similar discrete time intervals. Air quality data is optionally accompanied by the sensor assembly identification number, a site identification number and a time/data stamp when the air quality data was collected.

The controllers 158, 174 organize the air quality data to provide a comprehensive picture of the air quality attributes for the site 124. The controller 158 stores the air quality data in the database 160 using a variety of techniques. In one embodiment, air quality data is added to the database as a rolling average over a particular time interval (e.g., five minutes).

Data can be retrieved by the data collection program and dedicated computer at a regular interval, adjustable by editing the “time interval” field in a database that controls operation of the collection computer. Discrete “snapshot” values can be averaged together to compute an average value for all five parameters monitored. The master database is preferably updated periodically as soon as a new average has been computed, typically within a few seconds of having retrieved the data, such as for example via the Internet.

Customers can access the data through an Internet connection and see trend charts of average, hourly or daily values extending back in time over predetermined intervals. Customers can also view the instantaneous (updated once every 20 seconds in this example) data value for each sensor assembly through another custom designed interface called the real-time viewer. In one embodiment, records of five-minute averages for all sites and sensors are retained indefinitely.

Historical data going back a year or more is preferably available to the customer through the Internet interface at any time. To manage data access more effectively and speed up customer access to the data records, historical data are maintained in two separate databases: one containing all data for the last month, a second for all data older than 32 days. A custom-built software program and sensing logic automatically and transparently routes a customer request for data older than 32 days to the second database. Most often the typical customer is interested in recent data. Thereby the processing power of the web server can be devoted to the much smaller database of 32 days for the majority of customer transactions. This allows maximum processing speed and minimum access time.

If any of the air quality data exceeds predetermined thresholds, an automatic alert can be sent using a variety of techniques. In one embodiment, an automatic e-mail is sent from the private communications network 150 over the public communications network 130 to a remote user 180 and/or to any user in the private communications network 122. In another embodiment, the controller 158 can initiate an automated call to a telephone or a pager through the public communications network 130. In yet another embodiment, an alert signal is sent by the controller 158 through the public communications network 130 to an alert device 182 connected to the private communications network 122 at the site 124.

In an alternate embodiment, air quality data collected by the sensor assemblies 140 is stored and/or processed on the private communications network 122 at the site 124. For example, the workstation 126 can include a controller 196 and database 198. The workstation 126 can process the air quality data and maintain the database 98 substantially as done at the second private communications site 150. The database 198 can be accessed through the private communications network 122, such as from workstation 128 and/or at a remote site 180 through the public communications network 130. The workstation 126 may optionally be coupled through communication network 22 to other workstations such as 142 and 144, as shown in FIG. 5, or may be a stand-alone device. It is contemplated that such “on site” air-quality monitoring systems would be appealing to sophisticated end-users such as large hotels, office buildings or other large commercial spaces. Such end users will have the facilities maintenance staff resources required to perform frequent, real-time or near real-time analysis of the generated air quality data.

In another alternate embodiment, air quality data collected by the sensor assemblies 140 is stored on both the private communications network 122, such as on workstation 126 and on the second private communications network 150. The redundancy provided by the databases 160 and 198 may obviate the third private communications network 170.

The site 124 may also be connected to various utility providers 90, 92 such as electric, natural gas, telecommunications, security, and the like. In another alternate embodiment, the cost of the present air quality monitoring system 120 is billed to the site 124 along with the utility services. This embodiment takes advantage of the billing infrastructure of the utility providers 190, 192.

Thus, as described, the air quality monitoring system 120 of the present invention assures that air quality is automatically and systematically monitored without reliance upon schedules or priorities of personnel or individuals at the site 124. The air quality data collected by the sensor assemblies 140 is analyzed to control air quality or may be used for maintaining air quality records. For example, the data may be used to determine the frequency at which filtering devices, which are used to filter residues from the air, need to be changed.

FIG. 6 is a schematic illustration of a sensor assembly 200 in accordance with the present invention. The sensor assembly 200 includes a microprocessor 202 operatively coupled to a memory storage device 204, such as a read/write semiconductor device. The sensor assembly 200 illustrates the preferred configuration of a sensor array of such as those shown in FIG. 5 and designated collectively as 140. The storage device 204 preferably has sufficient capacity to store security protocols, to accept upgrades to the operating software, to maintain calibration data for the various sensors, and to maintain software for converting sensor data to a form usable by either the communications interface 242 or the private communications network 222. The memory storage device 204 can optionally have sufficient capacity to retain sensor data and/or air quality data for some period of time.

In the illustrated embodiment, the sensor assembly 200 includes a variety of sensors, such as digital thermometer 210, analog humidity sensor 212, analog odor and gases sensor 214 (e.g., VOC), analog CO sensor 216 and digital CO₂ sensor 218. The temperature sensor 210 and the humidity sensor 212 are preferably isolated from the other sensors by thermal barrier 220. The sensors 210, 212, 214, 216, 218 preferably continuously measure levels of the target air quality attribute, not just thresholds. Consequently, trends in air quality data can be tracked, as is discussed below. The entire sensor assembly 200, including all of the sensors 210, 212, 214, 216, 218, is preferably located on a single printed circuit board 221.

Sensor data generated by the analog sensors 212, 214, 216 is typically a voltage signal proportional to the measured level of an air quality attribute. Analog sensor data is preferably converted to digital sensor data by analog-to-digital converter (A-to-D) 222 for use by the microprocessor 202. The digital sensors 210, 218 are directly coupled to the microprocessor 202. The microprocessor 202 converts raw sensor data to air quality data. The conversion to air quality data units is accomplished by custom software that references individual sensor specific calibration data. That is, test data that relates the sampled information, in this case analog to digital converter counts, to a known level of the parameter of interest.

The air quality data is then preferably converted by the microprocessor 202 to an appropriate communications protocol. However, it is contemplated that the raw digital data may be transmitted to the communication system to 222 with further processing downstream. Turning back to the preferred embodiment, communications driver 224 transmits the air quality data to communications interface 232 and then to the private communications network 222 and/or an HVAC controller 234. In one embodiment, the communications interface 232 is compatible with a LAN protocol, such as Ethernet protocol. An HVAC controller is typically a programmable logic controller or other programmable device that controls the operation of various HVAC equipment. In another embodiment, the communications driver 224 transmits the air quality data directly to the HVAC controller 234. In yet another embodiment, the air quality data is transmitted through the private communications network 222 to the local HVAC controller 234. Since the communications driver 224 transmits digital data, the air quality data can be sent long distances with minimal interference from unwanted voltages or currents (i.e., noise).

Various sensors may be employed for measuring air velocity, dew point, air pressure, and/or the level of concentration of one or more undesirable gases in the ambient air, such as sulfur dioxide, methane, ammonia, propane, and the like. Sensors that measure the level of particulates, such as dust, aerosol droplets, bacteria, spores, pollen, and viruses may also be used. For particulate detecting, an ionization detector or back scattering infra-red detector may be employed. An ionizing smoke or particle detector is commercially available from Dicon Safety Products, Inc. of Toronto, Ontario, Canada and can be adapted for use as a sensor by modifying the device to output a voltage proportional to the particles detected by the electrodes. Other sensors that produce an electronic signal proportional to the level of foreign substances present in the ambient air, such as toxins, molds, or other chemicals, may be employed and the invention is not intended to be limited to the particular sensors described. Suitable additional sensors are disclosed in U.S. Pat. No. 5,255,556 (Lobdell).

In one embodiment, the odor and gases sensor 214 provides a relative indication of air quality, without identifying particular VOC's present in the air. A broadband odor and gases sensor permits more cost-effective detection of VOC's. In one embodiment, the odor and gases sensor 214 is calibrated using a reference gas, such as toluene. For example, 0-100 ppm of toluene corresponds to 0-100% of the permitted level of VOC's. All VOC's detected by the sensor 214 are then combined and converted to a single indication of relative air quality on the scale of 0-100%.

Some of the sensors, such as the odor and gases sensor 214 and the CO sensor 216, typically need to be heated in order to operate accurately. Power regulator 228 provides current to resistance heaters in each of the sensors 216 and 218. The microprocessor 202 controls a digital potentiometer 230 that sets the proper level of heater operation. Additional circuitry then automatically re-adjusts the voltage supplied to the heater resistances of the odors and gasses, and CO sensors 214, 216 to continuously and accurately maintain the desired temperature. The desired temperature can be determined empirically or using data provided by the sensor manufacturer. Accurate control of the temperature prevents temperature changes from occurring and being interpreted as changes in the odor and gases or CO levels.

In another embodiment, the microprocessor 202 combines raw sensor data (e.g., voltages) and/or air quality data from two or more sensors to generate a composite air quality index. The present air quality index is a composite number that factors in the interdependency of various air quality attributes. For example, odors and gases are worse in the presence of higher humidity. A composite air quality index based upon the odors and gases sensor and the humidity sensor will more accurately reflect the true impact of the odors and gases than separate data for each of these sensors. Similarly, increased levels of CO₂ are more problematic at higher temperatures. Again, a composite air quality index that based upon data from the C CO₂ and temperature sensors will more accurately reflect the impact of CO₂. In one embodiment, data from all of the sensors are combined into a single air quality index. In yet another embodiment, the controller 58 at the second private communications network 50 combines air quality data from two or more sensors to generate the air quality index.

In another embodiment, memory device 204 has sufficient capacity to store sensor data and/or air quality data for a discrete period of time. In the event that the communications link between a sensor assembly 200 and the second private communications network 250 fail, the sensor assembly 200 is capable of retaining the sensor data and/or air quality data for a period of time, such as for example seven days, until the connection is reestablished. Once the connection is reestablished, the microprocessor 202 downloads the stored air quality data to the private communications network 222 for processing as discussed herein.

In one preferred method of the invention, the air quality monitoring system may be used to monitor for illicit smoking on the premises and, may also be used, to take action to further action to prevent further illicit smoking. In this method, one or more of the sensors 210, 212, 214, 216 monitor for detectable byproducts of cigarette smoking such as, for example, elevated carbon monoxide levels, ammonia levels, formaldehyde levels, and/or hydrogen cyanide levels in a designated non-smoking room are space. One or more of the microprocessor 202, HVAC controller 234, or workstation coupled thereto by communication network to 222 are programmed to recognize air quality levels of one or more of such byproducts which are indicative of indoor tobacco smoking. One or more of the microprocessor 202, HVAC controller 234, or workstation coupled thereto by communication network to 222 generate a smoking detected signal when the presence of one or more tobacco smoke byproducts exceeds the predetermined levels. The smoking detected signal may be then communicated to facilities operations staff to notify them that a guest is suspected to be illicitly smoking in a designated non-smoking room or space. In an alternate embodiment of the method, the smoky detective signaled may be transferred to a communication network coupled to the facility's billing computer system. The facility's billing computer system is programmed to generate a record of the suspected elicit smoking event. The facility's billing computer system can then match the record to a database containing information concerning the guest currently occupying the designated non-smoking room or space in question. The facility's billing computer system is further program to query a customer database for prior instances of suspected illicit smoking. The facility's billing computer system may also be programmed to take the further step of (a) charging the account of the suspected illicitly smoking guest or tenant for an additional room cleaning charge to removing any unpleasant orders, (b) banning the suspected illicitly smoking guest or tenant in question from reserving or occupying non-smoking rooms in the future, and/or (c) the further step of recording the suspected illicit smoking incident in the customer database.

Another preferred method of the invention, utilizes both the indoor air quality monitoring system and the indoor air quality purification systems set forth above. The air quality monitoring system is provided with a sensor capable of detecting air quality data which is indicative of the presence or absence of a human being within the interior space. Preferably, the carbon dioxide sensor 218 is used to detect increased in carbon dioxide levels in the space. The air quality monitor system is coupled to an air purification system 122 by controller 234 or 134. The indoor air quality purification system 122 preferably has at least one sanitizing mode of operation, such as when relatively high concentration of ozone is generated, during which the presence of humans in the interior space is undesirable. The controller 134 or 234 is programmed to selectively activate and de-activating the sanitizing mode of the air quality purification system 122 in response to the air quality data which is indicative of the presence or absence of humans within an interior space. The preferred switching component of the system is preferably a microprocessor (not shown) within the controller 134 or 234, but may also be a mechanical switch. It is also prefer that the air quality purification system 122 include at least one gentler, air purification mode in which humans may be present within the interior space during operation of the mode. Preferably, the air purification system 122 of this embodiment of the invention includes an ozone generator which may be switched between sanitizing and purification modes. The low ozone output mode which would be at a level that is more appropriate for human occupation of the interior space during operation, but which would be less efficient at removing airborne contaminants from the space than in the sanitizing mode. In this way, after an interior space is vacated by its human occupants, the air purification system can automatically be switched to sanitation mode so that stronger odors, airborne particles, bacteria, mold, viruses can be neutralized by the system.

Where an automatic air quality analysis system is not available, it is preferred that the interior space of the invention be re-inspected and tested every six months to ensure that the maintenance procedures have been correctly followed and that the interior space has maintained acceptable chemical emissions, common allergen counts and, if a bio-aerosol baseline has been established for the space, its bio-aerosol content. The inspection should include visual inspections of the filters in the ventilation system, air purifications system (if a separate unit is provided), activated charcoal filters, HVAC (if present), and HEPA vacuum cleaner. Air samples should be taken for particulates, chemical emissions, including total VOCs, as well as problematic chemicals and irritants. The results of this testing should be compared to the baseline levels recorded for the interior space. It is expected that some variation in quantities of particulates and chemicals results will occur overtime. However, it is expected that variations will be larger for particulate allergens since mold spore counts and pollen contents can very greatly in the outdoor environment depending on the season and weather conditions. To determine if measured indoor variations are cause for concern, it is beneficial to compare the results for the indoor space with the outdoor environment, and preferably, also with samples taken from similarly situated indoor spaces that have not been treated with the allergy friendly methods of the invention. These comparisons should show whether a variation is due to high allergen of chemical readings in the outdoor environment or whether the staff may have failed to follow the proper maintenance procedures. Also, if there had been any water leeks or moisture build up within the room, unusually high mold counts should bring such a condition to the attention of the HIC conducting the inspection and review. Of course, if extensive mold growth was found, it would require remediation in accordance with the methods described above. Assuming bio-aerosol baselines have been set, elevated levels of the same bio-aerosols previously found in the space will typically be cause for concern since it may indicate that the source of the bio-aerosol was not properly remediated or that the problematic organisms are again actively growing in the space.

Assuming that the interior space is at or near the baseline values measured when the interior space was first tested, it is preferred that a dated certificate stating this be generated and that it be maintained in the records of the property owner. The maintained records for each testing occasion should include weather conditions and any comparison testing (outdoor or similar control rooms) that was conducted at the same time. These records should be retained so that the HIC doing future reviews and testing can analyze the historic records to compare any current variations with past ones that might be due to the seasonal or weather factors. These insights should be helpful in determining whether current variations are due to issues within the interior space or due to exterior factors that are beyond the control of the property owner.

1. A method of improving the air quality of an existing interior space including the steps of:
sampling the interior space for chemical pollutants and other common allergen content; analyzing the chemical pollutant and other common allergen content of the samples;
removing suspected sources of the allergens, chemical pollutants and bio-aerosols in the interior space;
selecting replacement interior materials with chemical pollutant and other common allergen content below predetermined acceptable values;
utilizing the selected materials within the interior space; testing samples of the completed interior space for chemical pollutant and other common allergen content to set an air quality base line for the interior space; and,
maintaining the interior space in a manner which the chemical pollutant and other common allergen content are kept at or near the air quality baseline.

2. The method of claim 1 further including the step of monitoring the indoor air quality of the space on a continuous or a nearly continuous basis to provide real-time feedback for the facilities management staff as well as for training cleaning crews and maintenance personnel.

3. The method of claim 1 further including the step of periodically testing the interior space samples for chemical pollutant and other common allergen content to evaluate whether the maintenance process has been effective in keeping the chemical pollutant, and other common allergen content of the space at or near the air quality baseline level.

4. The method of claim 1 further including the step of selecting materials that are less conducive to the growth of biological organisms that commonly produce potential allergens or bio-aerosols.

5. The method of claim 1 further including the step of sampling and analyzing for bio-aerosols.

6. The method of claim 1 further including the step reconstructing the interior space and retesting for potential allergens or bio-aerosol.

7. The method of claim 1 further including the step of maintaining the low chemical pollutant, bio-aerosol, and other common allergen content of the interior space by utilizing a suitable air purification system.

8. The method of claim 1 in which the suitable air purification system includes air purification sub-systems.

9. The method of claim 1 further including the step of field testing at least some of the materials to be brought into the interior space to ensure that they meet the manufacturers' specifications for low VOC off gassing and being free of common chemical irritants.

10. The method of claim 1 further including the step consulting with furniture, carpeting and other interior material manufacturers to assist in the selection of the chemicals and materials used in manufacturing the furniture, carpeting and other interior furnishing

11. A method of the constructing a low VOC, low allergen, and low bi-aerosol interior space including the steps of:
selecting interior buildings materials which total VOC off gassing is less than about 0.5 mg/m3;
selecting structural supports which total VOC off gassing is less than about 0.5 mg/m3;
selecting interior wall materials which total VOC off gassing is less than about 0.5 mg/m3;
selecting flooring materials which total VOC off gassing is less than 0.5 mg/m3;
and selecting furnishings which total VOC off gassing is less than about 0.5 mg/m3.

12. The method of claim 11 further including the step of testing the constructed interior space to set a base line for VOC content.

13. The method of claim 11 further including the step of training staff to maintain the air quality of the space at or near that baseline.

14. An interior space having improved air quality comprising: structural supports selected to total VOC off gassing is less than about 0.5 mg/m3; interior wall materials selected to total VOC off gassing is less than about 0.5 mg/m3; flooring materials selected to total VOC off gassing is less than about 0.5 mg/m3; and furnishings selected to total VOC off gassing is less than about 0.5 mg/m3

15. A system of constructing and maintaining the indoor air quality of an interior space including the steps of:
selecting construction materials with chemical pollutant and other common allergen content below predetermined acceptable values;
constructing the space utilizing the selected materials within the interior space;
near continuous monitoring of the indoor air quality of the completed interior space for chemical pollutant and/or other common allergens;
analyzing the indoor air quality data for elevated levels on chemical pollutants and/or other common allergens;
and remediating any suspected sources of elevated levels of chemical pollutants and/or other common allergens in real time.

16. A method detecting elicit smoking in a nonsmoking space including the steps of:
near continuous monitoring of the indoor air quality of a guest space for byproducts of tobacco smoking;
analyzing the indoor air quality data for tobacco smoking byproducts above a predetermined level;
generating a signal when the presence of one or more tobacco smoke byproducts exceeds the predetermined level;
and communicating that signal to facilities operations staff to notify them that a guest is suspected to be illicitly smoking in a non-smoking guest room.

17. The method of claim 16 further including the step of generating a record of the suspected elicit smoking event, matching the record to a database containing information concerning the guest currently occupying the room in question, and querying the database for prior instances of suspected illicit smoking.

18. The method of claim 16 further including the step of charging the account of the suspected illicitly smoking guest for an additional room cleaning charge to removing any unpleasant orders and/or banning the suspected illicitly smoking guest in question from occupying non-smoking rooms in the future.

19. A system for maintaining the indoor air quality of an interior space comprising,
a real-time indoor air quality monitoring sensor capable of detecting air quality data which is indicative of the presence or absence of a human being within the interior space,
air purification system operably connected to the real-time indoor air quality sensor, the indoor air quality purification system having at least one sanitizing mode of operation during which the presence of humans in the interior space is undesirable,
and a switching component for selectively activating and the de-activating the sanitizing mode of the air quality purification system in response to the air quality data which is indicative of the presence or absence of humans within an interior space.

20. The method of claim 1 further including the step of selecting a minimum air quality standards for a particular type of existing interior space, the minimum air quality standards including airborne particulate content, and further including the step of monitoring the indoor air quality of the space on a nearly continuous basis to ensure that the selected air quality standard is being maintained for the space over an extended period of time.
